(19) United States
(12) Patent Application Publication
(10) Pub. No.: US 2004/0131665 A1
(43) Pub. Date: Jul. 8, 2004

(54) Topical anesthetic formulation

(76) Inventor: Scott T. Wepfer, Hoover, AL (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/645,951

(22) Filed: Aug. 22, 2003

Related U.S. Application Data
(63) Continuation-in-part of application No. 10/111,241, filed on Jul. 10, 2002.
Application No. 10/111,241 is a 371 of international application No. PCT/US00/41451, filed on Oct. 23, 2000.
(60) Provisional application No. 60/161,155, filed on Oct. 22, 1999.

(51) Int. Cl. A61K 9/70; A61K 31/24

(52) U.S. Cl. 424/449; 514/536

(57) ABSTRACT

The topical anesthetic gel formulation of the present invention preferably includes lidocaine, USP as the active anesthetic ingredient with a skin penetration enhancer and a gelling agent. This invention deals with problems commonly associated with topical application of local anesthetics such as: slow onset of action; need for occlusion; messiness of creams, ointments or gels; and rapid loss of effect due to rapid systemic dispersion. The invention permits enhanced penetration of the anesthetic and thereby allows for a lesser total dosage of pharmaceutically active ingredient. The use of a lesser total dosage also decreases systemic toxicity.

### RELATED APPLICATIONS

This application is a Continuaton-In-Part of U.S. application Ser. No. 10/111,241 filed Jul. 10, 2002, now pending.

U.S. application Ser. No. 10/111,241 filed Jul. 10, 2002 is a US National Phase of PCT application number PCT/US00/41451 filed Oct. 23, 2000, now abandoned.

PCT application number PCT/US00/41451 filed Oct. 23, 2000 is a non-provisional of U.S. application No. 60/161,155 filed Oct. 22, 1999, now abandoned.

### FIELD OF THE INVENTION

The present invention generally relates to topical anesthetics. More particularly, the present invention relates to a fast acting topical anesthetic or transdermal pain formulation for deep dermis anesthesia for use prior to and/or during medical procedures.

### BACKGROUND OF THE INVENTION

The use of topical or dermal anesthetics has long been utilized in the practice of medicine. Topical anesthetics are commonly administered prior to procedures such as injections, biopsies, the application of laser energy for cutaneous procedures such as removal of hair, tattoos, telengectasias, etc., minor superficial surgeries, and the like.

One particular topical anesthetic utilized to suppress or eliminate pain during such procedures is known by the trade name EMLA®. This product is known to be effective as a topical anesthetic; however, EMLA® has a very long onset time, which is the time between administration of the topical anesthetic and the commencement of the anesthetic effect. It must also be covered with an occlusive dressing to enhance penetration. The onset time for EMLA® can range from 45 to 90 minutes and, in some instances, can take even longer. The variability in length of onset time leads to delays in the commencement of medical procedures and, because of the very wide variation in onset time, can lead to the premature commencement of procedures, thereby inflicting unnecessary pain on the patient.

Several topical anesthetic formulations have been extensively used by the medical field to obtain local anesthesia. These products are known to be effective as topical anesthetics; however, they typically have long onset times, which is the time between the administration of the topical anesthetic and the commencement of the anesthetic effect. They must also be covered with an occlusive dressing to enhance penetration. Also, the onset of action for these available topical anesthetics varies over a range of time, for example from 45 to 90 minutes. This variability in length of onset time leads to delays in the commencement of medical procedures and, because of the very wide variation in onset time, can lead to the premature commencement of procedures, thereby inflicting unnecessary pain on the patient. These current methods have used more viscous semi-liquid carriers such as creams, ointments or gels which can be quite messy to work with, which adds another inconvenience to the user. For example, they must be cleaned off the injection site before injecting.

Accordingly, it would be advantageous and desirable to develop a topical anesthetic formulation which has a shorter onset time, which has less variability in the onset time, does not require occlusion, is easier to apply with less mess and which is amenable to use for cutaneous laser procedures such as hair removal and skin resurfacing, as well as for use before giving injections, starting IVs, drawing blood, biopsies and minor superficial surgeries. Such a formulation will have a potent clinical use with a more rapid onset of action.

The ideal vehicle for such a formulation would enhance the percutaneous penetration of the active ingredient, allowing for a fast onset of action. At the same time, the active ingredient must not penetrate so effectively through the skin as to be rapidly lost to the systemic circulatory systems. Thus, the ideal vehicle would also enhance the skin's ability to retain the pharmacologically active ingredient or, in other words, to increase skin residence times.

### BRIEF SUMMARY OF THE INVENTION

The present invention concerns a topical anesthetic formulated as a gel for topical administration to the surface of the skin and into the deeper regions of the dermis. The topical anesthetic gel formulation of the present invention preferably includes lidocaine, USP as the active anesthetic ingredient. Additional constituents illustratively include a skin penetration enhancer and a gelling agent.

The invention confronts the paradoxical requirement that a local anesthetic quickly penetrate into the skin and produce a rapid onset of action, yet not penetrate into the systemic circulation. Also, the anesthetic does not have an adversely prolonged effect.

The present invention permits enhanced penetration of the anesthetic and thereby allows for a lesser total dosage of pharmaceutically active ingredient. The use of a lesser total dosage also decreases systemic toxicity.

A method for reducing pain sensation is provided that includes the step of applying a therapeutically effective amount of a first anesthetic formulation to an area of a patient's skin, mucosal tissue, or other surface area to be anesthetized. The first anesthetic includes a topical anesthetic compound and a skin penetration enhancer and a volatile co-solvent.

In a further step, a therapeutically effective amount of a gel anesthetic formulation is administered to the same area to be anesthetized. The gel anesthetic is allowed to remain in contact with the subject area for a period of time sufficient to reduce pain sensation.

### DETAILED DESCRIPTION OF THE INVENTION

The present invention provides a topical anesthetic formulation for topical administration to the surface of the skin and into the deeper regions of the dermis. The topical anesthetic formulation of the present invention is typically a solution which includes lidocaine, USP; benzyl alcohol, NF, anhydrous, isopropyl alcohol, USP.

Lidocaine, USP is the preferred active anesthetic ingredient. Advantages include its time to onset of action which is 0.5 to 1 minute. Another advantage of lidocaine is that methemoglobinemia is not a concern as it is in formulations which contain prilocalne.

The base or un-ionized form of this drug was intentionally chosen because it is significantly more soluble in benzyl alcohol and also because studies show that bases of local anesthetics more easily traverse the stratum corneum than do their salts. Lipid solubility appears to not only be the primary determinant of intrinsic anesthetic potency, the onset of action is also directly related to the percent of drug that exists in the base form since it is unchanged for that is primarily responsible for diffusion across the nerve sheath.

The key to this non-aqueous solvent and transdermal penetration system is benzyl alcohol. Benzyl alcohol has demonstrated its ability to not only solvate lipophilic (non-ionic) compounds, but to form a micelle, a property conducive to penetration of the stratum corneum. The high lipid solubility of lidocaine base as well as that of the benzyl alcohol greatly diminishes the need for a vasoconstrictor to be added to the formula to prolong the duration of anesthesia. Thus, the lipophilic nature is seen as a positive quality since vasoconstrictors are also contraindicated for many of the procedures for which this system will benefit, such as starting an IV and laser removal of telengiectasias. In both of these instances, vasoconstriction decreases the chances for success of the medical procedure.

The amphoteric properties of benzyl alcohol—its strong lipophilicity and moderate hydrophilicity—allow it to disrupt the highly structured lipid portion of the stratum corneum, or fluidizing its lipids, thus allowing lipid soluble drugs to pass through the stratum corneum at increased rates of absorption. It is then the same strong lipophilicity which enhances penetration that also significantly enhances the retention of lipophilic drugs in the subcutaneous tissues underlying the site of application, thus increasing the duration of local action and decreasing systemic side-effects by slowing continued penetration into the systemic circulation. Thus, more anesthetic molecules are allowed to reach the nerve membrane which improves the depth and duration of anesthesia.

Besides being an anesthetic itself, its ability to fluidize membranes may also play a role in the system's ability to bring about such a markedly fast onset of action.

The isopropyl alcohol is used as a co-solvent. Once applied to the skin, this co-solvent rapidly evaporates from the skin due to its greater volatility. As this happens, the drug is transferred to the less volatile phase, benzyl alcohol, which, due to its very rapid permeation and good solvent characteristics, prevents the deposition of solutes on the skin surface.

It is appreciated that other topical anesthetic compounds are operative herein in place of the above active anesthetic. Alternative topical anesthetic compounds illustratively include bupivacaine, chloroprocaine, oxyprocaine, mepivacaine, piperocaine, tetracaine, procaine, dibucaine, benzocaine, dyclaine and salts thereof. It is also contemplated that the present invention can optionally include a vasoconstrictor. Phenylephrine is a representative vasoconstrictor which could be utilized to keep the active ingredients localized to the site to which they are applied. Other vasoconstrictors could include naphazole, tetrahydrozoline, oxymetazoline, tramazoline, and salts thereof.

The addition salts of these compounds can be utilized in the formulation of the present invention. The benzyl alcohol serves as a penetration enhancer to allow deeper layers of the dermis to be anesthetized. The isopropyl alcohol serves as a co-solvent.

Typical ranges of the present invention are provided in Table I.

TABLE I
Typical Composition Ranges for Inventive Topical
Anesthetic in Total Weight Percent of the Formulation

| | | Typical | Preferred |
|---|---|---|---|
| Agent | Component | Range Values | Range |
| Vasoconstrictor (total) | | 0.05-5 | 1-3 |
| | phenylephrine HCl | 0.05-5 | 1-3 |
| Anesthetic (total) | | 1-25 | 5-16 |
| | procaine HCl | 0-15 | 0.5-4 |
| | lidocaine HCl | 0-20 | 0.5-6 |
| | tetracaine HCl | 0-25 | 1-9 |
| Skin Penetration | | | |
| Enhancer (total) | | 0-35 | 5-21 |
| | benzyl alcohol | 0-35 | 1-10 |
| | propylene glycol | 0-35 | 3-14 |
| VOC and base | | 40-99 | 70-90 |

It is appreciated that a variety of skin penetration enhancers, skin compatible and anesthetic solvating VOCs and bases in addition to those detailed herein are known to one skilled in the art. Skin penetration enhancers additionally operative here in place of or in combination with those of Table I illustratively include ethoxydiglycol and those detailed in “Percutaneous 15 Penetration Enhancers: The Fundamentals,” E. W. Smith and H. I. Maibach, July 1999, pp. 1-512, which is incorporated herein by reference. Additionally, a volatile organic compound intended to enhance evaporation such as isopropyl alcohol, an ether or halocarbon is optionally omitted in instances where rapid evaporation is not desired.

In use, a therapeutically effective amount of the topical anesthetic formulation of the present invention is applied to the skin of a patient or subject prior to and/or during a medical procedure to treat the patient or subject.

The terms “patient” and “subject” mean all animals including humans. Examples of patients or subjects include humans, cows, dogs, cats, goats, sheep, and pigs.

The term “treating” includes, but is not limited to, the application of the topical anesthetic to the skin of a patient to prevent or inhibit the sensation of pain in the vicinity or region of the application of the topical anesthetic formulation.

A therapeutically effective amount is an amount of the topical anesthetic formulation of the present invention, that when administered to a patient or subject, ameliorates, eliminates and/or inhibits pain in the local region or vicinity of the application of the topical anesthetic of the present invention.

Dosage forms for topical administration of the formulation of the present invention include creams, gels, ointments and topical sprays. The active components are admixed with a physiologically acceptable carrier and any preservatives, buffers, or propellants as may be required. Ophthalmic formulations, eye ointments, powders, and solutions, as well as dental formulations containing appropriate flavors and sweeteners, are also contemplated as being within the scope of this invention.

The topical anesthetic or transdermal pain formulation of the present invention can be packaged in a spray bottle or other suitable delivery device and can be applied to the surface of the skin utilizing a cotton swab, gauze pad, or other suitable applicator. A preferred formulation of the present invention can be made by combining the following ingredients:

To make 30 ml:

| | |
|---|---|
| lidocaine, USP | 1.2 gm (active ingredient) |
| benzyl alcohol | 3.0 ml (penetration enhancer) |
| isopropyl alcohol | 8.0 ml (to aid in quick drying |
| | by evaporation) |

Mixing Instructions:

Weigh out first four ingredients.

Transfer to 100 ml beaker.

Add paraben-preserved water.

Stir until dissolved.

When dissolved, add benzyl alcohol, isopropyl alcohol and propylene glycol.

Stir until well mixed.

Dispose in sprayer bottle.

Applicants have found the formulation according to the present invention to be 100% effective in preventing any discomfort associated with the laser removal of hair using an Alexandrite Laser in twelve of twelve patients. In six of these instances, the procedure had been previously done once before utilizing EMLA® gel which was applied approximately ninety minutes prior to the initiation of the laser hair removal. In these six patients, their procedures had to be stopped prematurely due to patient discomfort. When the patients were re-lasered after pre-treating with the transdermal pain formulation of the present invention, none of these six patients reported any discomfort from the second procedure which was completed. One of the twelve patients or subjects was a male who had hair removed from his back. This is an interesting result because, of the different types of laser hair removal procedures, the removal of hair from the back is thought to be one of the most painful.

While the use of the transdermal pain formulation or topical anesthetic formulation of the present invention has been described for use in the laser removal of hair, Applicant contemplates other uses including use prior to laser skin resurfacing and other cutaneous laser procedures, use prior to injection or insertion of an intravenous needle such as for the initiation of an intravenous drip, use prior to other types of needle sticks such as IM injections, inoculations and blood drawing, or other suitable uses for topical or transdermal anesthesia which are well known to those skilled in the art.

As noted above, an inventive transdermal pain composition or topical anesthetic composition of the present invention may be in the form of a gel. A preferred gel formulation is an anhydrous preparation that includes a topical anesthetic compound, a skin penetration enhancer, and a gelling agent. A topical anesthetic compound, and a skin penetration enhancer, included in a gel formulation are generally those described above. A skin penetration enhancer is included at concentrations ranging from 5% to 95%, preferably 40% to 90% of the total weight of the gel composition. Further, multiple skin penetration enhancers may be included in an inventive gel preparation. Preferred skin penetration enhancers include benzyl alcohol, 2,(2-ethoxyethoxy)ethanol, and propylene glycol.

Propylene glycol and 2,(2-ethoxyethoxy)ethanol are each individually typically present at concentrations of 0% to 90% of the total weight of the gel composition. A preferred range for each of these skin penetration enhancers is 20% to 60% of the total weight of the gel composition. Further preferred is a composition including one or both of these skin penetration enhancers at a concentration ranging from 25% to 45% of the total weight of the gel composition.

Benzyl alcohol is included in an inventive gel composition at concentrations ranging from 0% to 90% of the total weight of the gel composition, preferably 5% to 20%.

Gel formulations are known in the art as semi-solids. An inventive topical anesthetic gel formulation includes a gelling agent compatible with the components of the topical anesthetic described herein. For example, cellulose polymers compatible with skin penetration enhancers and other ingredients of a detailed gel composition are operative in an inventive gel formulation. A preferred gelling agent is hydroxypropyl cellulose. Hydroxypropyl cellulose is generally available in grades ranging from about 5 cps to about 25000 cps. Generally hydroxypropyl cellulose ranging in viscosity from 500 cps to about 5000 at room temperature is included in an inventive composition at a final concentration ranging from about 0.2% to about 5%. Preferably, hydroxypropyl cellulose 1500 cps is included at a final concentration ranging from 1% to 2% of the total weight of the gel composition.

Further optionally included in an inventive gel formulation is a dispersing agent. A dispersing agent is generally included in a gel composition in order to aid in achieving a uniform mixture. Exemplary dispersing agents include glycerin. A dispersing agent is included at concentrations ranging from 0 to 40%, preferably 5% to 25% of the total weight of the gel composition. Alternatively, composition ingredients are dispersed by other methods, such as stirring, heating, sonication, combinations of these and other dispersal methods known in the art.

A preservative is optionally included in an inventive composition at a concentration effective to inhibit undesirable effects such as microbial growth, UV and/or oxygen-induced breakdown of composition components, and the like. A preservative operative in an inventive gel is any of those known in the art and compatible with the components of an inventive composition. Examples include butylated hydroxytoluene (BHT) and edetate disodium. When a preservative is included, it is present at concentrations sufficient to confer a preservative effect, typically ranging from 0.01% to 1.5%, preferably 0.025% to 1%, depending on the preservative.

A fragrance is optionally added which may have the effect of pleasing and soothing the patient. An included fragrance is chosen which is compatible with the composition components. Menthol is an example of a suitable fragrance.

Other optional ingredients include, but are not limited to, a skin soothing agent, a coloring agent, a buffering agent, a film forming agent, an opacifying agent, a VOC, and a combination of any of these or other components known in the art to be typical in topical formulations. The total concentration of such “other” agents generally ranges between 0% to 20% of the total weight of the composition.

TABLE II
Typical Composition Ranges for Inventive Topical
Anesthetic Gel in Total Weight Percent of the Formulation

| | | Typical | Preferred |
|---|---|---|---|
| Agent | Component | Range Values | Range |
| Vasoconstrictor | | 0-5 | 1-3 |
| (total) | | | |
| | phenylephrine HCl | 0-5 | 1-3 |
| Anesthetic (total) | | 1-25 | 3-16 |
| | procaine HCl | 0-15 | 0.5-4 |
| | lidocaine HCl | 0-20 | 0.5-6 |
| | tetracaine HCl | 0-25 | 1-9 |
| Skin Penetration | | | |
| Enhancer (total) | | 5-95 | 40-90 |
| | benzyl alcohol | 0-95 | 5-20 |
| | propylene glycol | 0-95 | 25-45 |
| | 2, (2-ethoxyethoxy) | 0-95 | 25-45 |
| | ethanol | | |
| Gelling agent (total) | | 0.1-20 | 1-5 |
| | hydroxypropyl | 0.2-5 | 1-2 |
| | cellulose | | |
| Dispersing agent | | 0-40 | 5-25 |
| (total) | | | |
| | glycerin | 0-40 | 5-25 |
| Preservative (total) | | 0-5 | 0.5-3 |
| | BHT | 0-1.5 | 0.025-1 |
| | edetate disodium | 0-1.5 | 0.025-1 |
| Fragrance | | | |
| | menthol | 0-3 | 0.05-1 |
| Other | | 0-20 | 0.05-10 |

Method of Topical Anesthetization

A gel formulation of a topical anesthetic according to the present invention is used separately or in conjunction with another anesthetic formulation.

Used separately, a gel formulation is applied to the area of the patient to be anesthetized. Generally, an anesthetic effect is apparent within 30 minutes.

In combination with another topical anesthetic formulation, a synergistic effect is achieved. In this embodiment it is preferred to use a liquid anesthetic formulation as detailed in Table I in conjunction with a gel formulation as detailed in Table II. In a first step, a therapeutically effective amount of an inventive liquid anesthetic formulation is applied to an area of the patient to be anesthetized. Preferably, the liquid anesthetic formulation is applied as a spray, although other forms of application will be recognized as operable in an inventive method. Following application of a liquid anesthetic formulation, a therapeutically effective amount of a gel anesthetic formulation is applied to the same area. The anesthetic formulations are allowed to act for a period of time sufficient to achieve the desired level of anesthesia. The level of anesthesia may be determined by any of various methods known in the art, including patient report in response to painful stimulus.

### EXAMPLES

### Example 1

Gel Formulation

propylene glycol 40%

ethoxydiglycol 40%

glycerin 20%

hydroxypropyl cellulose 1500 cps 1-2%

menthol 0.1%

butylated hydroxytoluene 0.05%

edetate disodium 0.05%

other 7.8-8.8%

### Example 2

Gel Formulation—Total Weight 100 gm

Propylene glycol, USP 36.6 ml (38 gm)

2,2′-Ethoxyethoxyethanol 38.4 ml (38 gm)

Benzyl alcohol, USP 9.6 ml (10 gm)

Glycerin, USP 6.7 ml (8.4 gm)

Lidocaine, USP (base) 4.0 gm

Hydroxypropyl cellulose 1500 cps 1.5 gm

Menthol 0.1 gm (100 mg)

Following is the Specific Gravity (gm/ml) of the liquid ingredients used in the formula above:

Propylene glycol, USP 1.037

2,2′-Ethoxyethoxyethanol 0.989

Benzyl alcohol, USP 1.045

Glycerin, USP 1.249

### Example 3

Exemplary Mixing Directions for Gel Formulation of Example 3—Total Weight 100 gm

1. Measure out Benzyl alcohol and transfer into beaker or other suitable mixing container.

2. Weigh out Lidocaine, USP, and Menthol and transfer into benzyl alcohol one ingredient at a time. Stir until dissolved and then add next ingredient.

3. Measure out propylene glycol and ethoxydiglycol and add into above mixture of lidocaine dissolved in benzyl alcohol.

4. Measure out glycerin and add into above mixture.

5. Weigh out hydroxypropylcellulose 1500 cps and slowly add to above mix with stirring.

6. Continue to stir above mix for at least 12 hours or until a uniform, clear gel has formed.

### Example 4

To Make 100 gm of Gel Anesthetic

Benzyl Alcohol 10.0 gm (10 ml) (Specific gravity 1.045)

Lidocaine, USP (base) 4 gm

Menthol, USP 0.1 gm

Butylated hydroxytoluene, NF (BHT) 0.05 gm

Propylene glycol, USP 35 gm (33.784.ml) (Specific gravity=1.036)

Diethylene glycol monoethyl ether, reagent 35 gm (35.389 ml) (Specific gravity=0.989)

Edetate Disodium, USP 0.05 gm

Glycerin, USP 99.5% Anhydrous 14.8 gm (11.849 ml) (Specific gravity 1.249)

Hydroxypropylcellulose, NF 1500 cps 1 gm

### Example 5

Exemplary Mixing Directions for Formulation of Example 4

1. Measure out Benzyl Alcohol into Mixing Container.

2. Dissolve Lidocaine, Menthol & BHT in Benzyl Alcohol.

3. Measure out Propylene glycol and ethoxydiglycol and add to #2.

4. Dissolve edetate disodium in #3. (This takes several minutes of constant stirring)

5. Measure out Glycerin and add to mixture when disodium edetate is completely dissolved.

6. Measure out hydroxypropylcellulose 1500 cps and slowly add through a 40-mesh sieve into the mixture resulting from step #5 while constantly stirring mixture.

7. Stir until hydroxypropylcellulose has uniformly gelled (usually needs to stir over night)

Notes:

1. Can add or subtract hydroxypropylcellulose to obtain desired thickness.

It will be recognized by one of skill in the art that reagents purchased as “anhydrous” sometimes contain small amounts of water. Thus, the term “anhydrous” as used herein is intended to mean substantially anhydrous.

A commercial kit including at least one topical anesthetic compound formulated as described herein is provided, together with instructions for use thereof as a topical anesthetic. Optionally, the commercial kit of may further include a second topical anesthetic formulated as described herein.

A topical anesthetic formulation as described herein may further contain a therapeutic agent which may augment or complement the anesthetic action and the goal of the therapeutic intervention. Suitable therapeutic agents include analgesics, antianxiety, antiarrhythmics, antibacterials, antibiotics, anticoagulants, anticonvulsants, antifungals, antihistamines, antiinflammatories, antivirals, bronchodilators, calcium channel blockers, cytotoxics and anticancer agents, cytokines, growth factors, immunosuppressives, muscle relaxants, psychotherapeutics, sympathomimetics, vasodilators, vitamins and other therapeutic agents such as those found in Remington: The Science and Practice of Pharmacy, 20th Ed., A. Gennaro, Ed.) 2000; Goodman and Gilman's The Pharmacological Basis of Therapeutics, 10th Ed., Hardman J G et al. Eds., 2001, McGraw Hill; and The Merck Index, 13th Edition, 2001, O'Neill M J et al. Eds., Merck & Co.

In particular, an anesthetic formulation as described herein may contain a therapeutic agent which is an anti-itch or antipruritic agent. Anti-itch agents include antihistamines, for example, alkylamines such as bromphenphiramine maleate, chlorpheniramine maleate and dexchlorpheniramine maleate; ethanolamines such as diphenhydramine HCl, carbinoxamine and clemastine fumarate; ethylenediamines, including pyrilamine maleate; phenothiazines such as promethazine HCl; piperidines such as cyproheptadine HCl; and other antihistamines such as the non-sedating compounds astemazole, loratadine, fexofenadine and cetirizine. Further anti-itch agents include cooling and soothing compounds such as camphor, thymol, calamine and crotamiton.

An exemplary composition according to the invention containing an anesthetic agent and an anti-itch agent, includes an alkylamine at a concentration ranging from 0.5-10% of the total weight of the composition. A preferred composition contains an alkylamine in amounts ranging from 0.75-3% of the total weight of the composition. For example, a preferred composition contains 0.5-5% diphenhydramine hydrochloride.

In formulating an inventive composition containing both an anesthetic agent and an anti-itch agent, the anti-itch agent may be added to the mixture at the same time as the anesthetic. In order to adjust the total volume to accommodate the volume of the anti-itch agent, the volume of one of the other ingredients is lowered. Typically, the volume of one or more of the skin penetration enhancers is lowered in an amount equal to the volume of the anti-itch ingedient. However, as will be evident to one of skill in the art, the volume of one or more of the other ingredients may be lowered in order to include the anti-itch agent at a desirable concentration.

A method of reducing pain and itch using a gel formulation according to the present invention includes use of the anesthetic/anti-itch gel formulation separately or in conjunction with another anesthetic formulation.

Used separately, a gel formulation is applied to the area of the patient to be anesthetized. Generally, an anesthetic effect is apparent within 30 minutes. An anti-itch effect may be almost immediate or apparent within minutes to hours depending on the agent.

In combination with another topical anesthetic formulation, a synergistic effect is achieved. In this embodiment it is preferred to use a liquid anesthetic formulation as detailed in Table I in conjunction with an anesthetic/anti-itch gel formulation as described herein. In a first step, a therapeutically effective amount of an inventive liquid anesthetic formulation is applied to an area of the patient to be anesthetized. Preferably, the liquid anesthetic formulation is applied as a spray, although other forms of application will be recognized as operable in an inventive method. It is recognized that the liquid form containing an anesthetic may also contain an additional therapeutic agent, such as an anti-itch agent. Following application of a liquid anesthetic formulation, a therapeutically effective amount of a gel anesthetic/anti-itch formulation is applied to the same area. The applied formulations are allowed to act for a period of time sufficient to achieve the desired level of anesthesia and/or anti-itch effect. The level of anesthesia may be determined by any of various methods known in the art, including patient report in response to painful stimulus. Similarly, anti-itch effect is generally gauged by patient report of effectiveness.

A commercial kit is provided by the present invention which includes a gel formulation containing an anesthetic and a therapeutic agent as described herein. Instructions for use thereof may also be included in the kit.

It is recognized that a gel formula as described herein is a useful medium for delivery of a therapeutic agent and formulations other than those containing an anesthetic are contemplated. In particular therapeutic agents that may be included in a gel formulated as described herein include analgesics, anxiolytic compounds, antiarrhythmics, antibacterials, antibiotics, anticoagulants, anticonvulsants, antifungals, antihistamines, anti-inflammatories, anti-itch compounds, antivirals, bronchodilators, calcium channel blockers, cytotoxics and anticancer agents, cytokines, growth factors, immunosuppressives, muscle relaxants, psychotherapeutics, sympathomimetics, vasodilators, vitamins, and combinations of these; other therapeutic agents are included such as those found in Remington: The Science and Practice of Pharmacy, 20th Ed., A. Gennaro, Ed.) 2000; Goodman and Gilman's The Pharmacological Basis of Therapeutics, 10th Ed., Hardman J G et al. Eds., 2001, McGraw Hill; and The Merck Index, 13th Edition, 2001, O'Neill M J et al. Eds., Merck & Co.

Generally, an inventive gel formulation is an anhydrous preparation that includes a therapeutic agent, a skin penetration enhancer, and a gelling agent. A therapeutic agent, and a skin penetration enhancer, included in a gel formulation are generally those described above. A skin penetration enhancer is included at concentrations ranging from 5% to 95%, preferably 40% to 90% of the total weight of the gel composition. Further, multiple skin penetration enhancers may be included in an inventive gel preparation. Preferred skin penetration enhancers include benzyl alcohol, 2,(2-ethoxyethoxy)ethanol, and propylene glycol.

Propylene glycol and 2,(2-ethoxyethoxy)ethanol are each individually typically present at concentrations of 0% to 90% of the total weight of the gel composition. A preferred range for each of these skin penetration enhancers is 20% to 60% of the total weight of the gel composition. Further preferred is a composition including one or both of these skin penetration enhancers at a concentration ranging from 25% to 45% of the total weight of the gel composition.

Benzyl alcohol is included in an inventive gel composition at concentrations ranging from 0% to 90% of the total weight of the gel composition, preferably 5% to 20%.

Gel formulations are known in the art as semi-solids. An inventive therapeutic agent gel formulation includes a gelling agent compatible with the components of the therapeutic agent described herein. For example, cellulose polymers compatible with skin penetration enhancers and other ingredients of a detailed gel composition are operative in an inventive gel formulation. A preferred gelling agent is hydroxypropyl cellulose. Hydroxypropyl cellulose is generally available in grades ranging from about 5 cps to about 25000 cps. Generally hydroxypropyl cellulose ranging in viscosity from 500 cps to about 5000 at room temperature is included in an inventive composition at a final concentration ranging from about 0.2% to about 5%. Preferably, hydroxypropyl cellulose 1500 cps is included at a final concentration ranging from 1% to 2% of the total weight of the gel composition.

Further optionally included in an inventive gel formulation is a dispersing agent. A dispersing agent is generally included in a gel composition in order to aid in achieving a uniform mixture. Exemplary dispersing agents include glycerin. A dispersing agent is included at concentrations ranging from 0 to 40%, preferably 5% to 25% of the total weight of the gel composition. Alternatively, composition ingredients are dispersed by other methods, such as stirring, heating, sonication, combinations of these and other dispersal methods known in the art.

A preservative is optionally included in an inventive composition at a concentration effective to inhibit undesirable effects such as microbial growth, UV and/or oxygen-induced breakdown of composition components, and the like. A preservative operative in an inventive gel is any of those known in the art and compatible with the components of an inventive composition. Examples include butylated hydroxytoluene (BHT) and edetate disodium. When a preservative is included, it is present at concentrations sufficient to confer a preservative effect, typically ranging from 0.01% to 1.5%, preferably 0.025% to 1%, depending on the preservative.

A fragrance is optionally added which may have the effect of pleasing and soothing the patient. An included fragrance is chosen which is compatible with the composition components. Menthol is an example of a suitable fragrance.

Other optional ingredients include, but are not limited to, a skin soothing agent, a coloring agent, a buffering agent, a film forming agent, an opacifying agent, a VOC, and a combination of any of these or other components known in the art to be typical in topical formulations. The total concentration of such “other” agents generally ranges between 0% to 20% of the total weight of the composition.

An example of a gel formulation containing a therapeutic agent includes a gel as described above containing an antibacterial agent. For instance, an antibacterial gel may be formulated as above except that the anesthetic is not included and an antibacterial agent is added. Antibiotics illustratively include aminoglycosides such as streptomycin, neomycin and gentamycin; cephalosporins such as cephalothin, cefazolin, cefalexin, cefuroxime, cefamandole, cefoxitin and cefaclor; antibiotic glycopeptides such as vancomycin; lincosamides such as clindamycin; macrolides such as erythromycin; nitroimidazoles such as tinidazole; penicillins such as azocillin, nafcillin, methicillin, ampicillin, amoxacillin; sulfonamides; tetracyclines; antibiotic polypeptides such as bacitracin; and quinolones such as ciprofloxacin. Other antibacterials are known in the art and more information on such compounds may be found in standard pharmacology references such as Remington: The Science and Practice of Pharmacy, 20th Ed., A. Gennaro, Ed.) 2000; Goodman and Gilman's The Pharmacological Basis of Therapeutics, 10th Ed., Hardman J G et al. Eds., 2001, McGraw Hill; and The Merck Index, 13th Edition, 2001, O'Neill M J et al. Eds., Merck & Co. An exemplary formulation includes an antibiotic selected from the group polymyxin B sulfate, bacitracin zinc and neomycin sulfate and combinations thereof. An antibiotic selected from this group is included at an appropriate dosage, for example, polymyxin B sulfate may range in amount from 1000-50000 units per gram of formulation, bacitracin zinc may range in amount from 100-5000 units per gram of formulation and neomycin sulfate may be added in amounts equivalent to about 1-25 milligrams of neomycin base per gram of formulation. A suitable mixture of antibiotics is illustrated by the combination of polymyxin B sulfate—10000 units per gram of gel formulation, bacitracin zinc—500 units per gram of gel formulation and neomycin sulfate equivalent to about 3.5 mg of neomycin base per gram of gel formulation. Other antibiotic formulations, combinations and concentrations may be included in a gel formulation as appropriate for the therapeutic application.

### Example 6

To Make 100 gm of Gel Antibiotic

polymyxin B sulfate 10000 units per gm

bacitracin zinc 500 units per gm

neomycin sulfate equivalent to 3.5 mg of neomycin base per gm

Butylated hydroxytoluene, NF (BHT) 0.05 gm

Propylene glycol, USP 35 gm (33.784.ml) (Specific gravity=1.036)

Diethylene glycol monoethyl ether, reagent 35 gm (35.389 ml) (Specific gravity=0.989)

Edetate Disodium, USP 0.05 gm

Glycerin, USP 99.5% Anhydrous 14.8 gm (11.849 ml) (Specific gravity=1.249)

Hydroxypropylcellulose, NF 1500 cps 1 gm

Skin Penetration Enhancers and/or “other” ingredients to 100 g.

A commercial kit is provided by the present invention which includes a gel formulation containing a therapeutic agent as described herein. Instructions for use thereof may also be included in the kit.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

In view of the teaching presented herein, other modifications and variations of the present invention will readily be apparent to those of skill in the art. The discussion and description are illustrative of some embodiments of the present invention, but are not meant to be limitations on the practice thereof. It is the following claim, including all equivalents, which defines the scope of the invention.

1. A formulation comprising at least one anesthetic compound selected from the group consisting of procaine, lidocaine, tetracaine and salts thereof; and a skin penetration enhancer, and a gelling agent in an anhydrous mixture.

2. The formulation of claim 1 wherein said skin penetration enhancer is at least one compound selected from the group consisting of: benzyl alcohol, propylene glycol, and ethoxydiglycol.

3. The formulation of claim 1 wherein at least two skin penetration enhancers are present.

4. The formulation of claim 1 wherein said at least one anesthetic compound is present in said formulation from 1 to 25 total weight percent.

5. The formulation of claim 3 further comprising a third skin penetration enhancer.

6. The formulation of claim 1 wherein said at least two anesthetic compounds are procaine, lidocaine and tetracaine, or salts thereof.

7. The formulation of claim 1 wherein lidocaine is present from 0.5-6 total weight percent.

8. The formulation of claim 1 wherein said skin penetration enhancer is present from 0 to 95 total weight percent.

9. The formulation of claim 1 wherein said skin penetration enhancer is present from 25 to 45 total weight percent.

10. The formulation of claim 1 wherein the gelling agent is a cellulosic polymer.

11. The formulation of claim 1 wherein the gelling agent is hydroxypropyl cellulose.

12. The formulation of claim 11 wherein the hydroxypropyl cellulose has a viscosity ranging between 1000 and 2000 centipoise at 25 degrees centigrade.

13. The formulation of claim 12 wherein the concentration of hydroxypropyl cellulose ranges from 0.5-5 total weight percent.

14. The formulation of claim 1 further comprising a dispersing agent.

15. The formulation of claim 1 further comprising an ingredient selected from the group consisting of: preservative, fragrance, buffer, and an emollient.

16. The formulation of claim 1 further comprising a therapeutic agent.

17. The formulation of claim 16 wherein the therapeutic agent is selected from the group consisting of: analgesics, anxiolytic compounds, antiarrhythmics, antibacterials, antibiotics, anticoagulants, anticonvulsants, antifungals, antihistamines, anti-inflammatories, antivirals, bronchodilators, calcium channel blockers, cytotoxics and anticancer agents, cytokines, growth factors, immunosuppressives, muscle relaxants, psychotherapeutics, sympathomimetics, vasodilators, vitamins, and combinations of these.

18. The formulation of claim 16 wherein the therapeutic agent is an anti-itch compound.

19. A method for reducing pain sensation, the method comprising the steps of:
applying a therapeutically effective amount of an anhydrous gel anesthetic formulation according to claim 1 to the area of an individual's skin to be anesthetized; and
allowing the gel anesthetic to remain in contact with the area for a period of time sufficient to reduce pain sensation.

20. The method of claim 19 further comprising the step of:
applying a therapeutically effective amount of an anesthetic formulation comprising a topical anesthetic compound, a skin penetration enhancer and a volatile co-solvent, to the area to be anesthetized.

21. A method for reducing pain associated with the application of laser energy to the skin, said method comprising the step of applying a therapeutically effective amount of a topical anesthetic according to claim 1 to the area of the skin to be treated prior to the application of laser energy.

22. A commercial kit comprising at least one topical anesthetic compound formulated according to claim 1, together with instructions for use thereof as a topical anesthetic.

23. The commercial kit of claim 22 further comprising a second topical anesthetic, said second topical anesthetic comprising a skin penetration enhancer and a volatile co-solvent.

24. A method of local anesthesia comprising the step of applying to intact skin or oral mucosa the formula of claim 1.
